(19) United States
(12) Patent Application Publication
(10) Pub. No.: US 2021/0341449 A1
(43) Pub. Date: Nov. 4, 2021

(54) METHOD OF VALIDATING A WATER DETERMINING DEVICE USING A ROOM TEMPERATURE IONIC LIQUID

(71) Applicant: HONEYWELL INTERNATIONAL INC., Charlotte, NC (US)

(72) Inventor: Roman Neufeld, Hannover (DE)

(73) Assignee: HONEYWELL INTERNATIONAL INC., Charlotte, NC (US)

(21) Appl. No.: 17/244,273

(22) Filed: Apr. 29, 2021

Related U.S. Application Data
(60) Provisional application No. 63/018,871, filed on May 1, 2020.

(51) Int. Cl. G01N 33/18

(52) U.S. Cl. CPC G01N 33/1813; G01N 33/182

(57) ABSTRACT

A method of validating a water determining device includes the steps of providing a liquid water standard including a room temperature ionic liquid, adding the liquid water standard to the water determining device, and determining the amount of water in the liquid water standard utilizing the water determining device thereby validating the water determining device.

### CROSS-REFERENCE TO RELATED APPLICATIONS

This application claims the benefit of U.S. Provisional Application No. 63/018,871 filed May 1, 2020, which is incorporated by reference herein in its entirety.

### TECHNICAL FIELD

The present disclosure generally relates to a method of validating a water determining device. The present disclosure more specifically relates to use of a liquid water standard that includes a room temperature ionic liquid that is stable at high temperatures.

### BACKGROUND

The determination of an amount of water in a sample is one of the standard methods of analytical chemistry. The water content can be determined in solid, liquid or gaseous samples. In these processes, quality management plays an important role especially relative to calibration, validation, and inspection of water determining devices. Typically, water standards that include known amounts of water are used as reference samples to calibrate and validate the water determining devices. Water standards are also used for titer determination, monitoring precision and accuracy of titrations, and validation and inspection of Karl Fischer titrators according to ISO, GMP, GLP and FDA guidelines.

Some titration techniques require both titer determination/standardization and periodic performance checks to monitor instrument accuracy and precision using standards appropriate to each task. While 100% water may theoretically be used, there are a number of practical advantages to using a certified water standard instead. The main disadvantage to using 100% water is that the required sample size is sufficiently small (10-20 mg) to require highly skilled handling in order to avoid errors related to sample weighing and transfer. Furthermore, laboratory water may not necessarily be free of potentially interfering contaminants. Finally, a number of regulatory bodies have recently begun to emphasize the need to have standards used in the laboratory be traceable to an appropriate national standard reference material. Moreover, the use of solid standards necessitates the opening of the titration cell to atmosphere, which risks contamination by atmospheric moisture. Additionally, finely ground solids tend to stick to greased sample ports and adhere to the walls of the titration cell due to static generated during their transfer. The use of sodium tartrate dihydrate, in particular, is further complicated by its limited solubility in methanol. This poor solubility necessitates limiting the sample size, which, again, can lead to errors related to sample weighing and transfer.

Other titration techniques can also require periodic performance checks to monitor instrument accuracy and precision since such factors as ambient temperature and humidity conditions, quality of titration cell seals and septa, freshness of desiccant in drying tubes, chemistry of the samples, and others may introduce error into the analysis. While 100% water may, again, theoretically be used for this purpose, the sample size and handling issues can be challenging. In some techniques that are semi-micro methods, the required sample size for 100% water is extremely small (100-500 μg), and weighing and sample transfer errors become even more difficult to avoid. Moreover, some laboratory water may not necessarily be free of potentially interfering contaminants, and electrochemical techniques are typically more sensitive to impurities that can change the conductivity of the system. Therefore, water standards are typically used.

Typically, it is recommended to use a water standard that is physically compatible with the sample to be analyzed. Therefore, a solid sample typically requires use of a solid water standard and a liquid sample typically requires use of a liquid water standard. Unfortunately, no liquid water standards are commercially available that are stable at elevated temperatures (e.g. at temperatures greater than 150° C.).

There is a need to develop liquid water standards that are stable at elevated temperatures for a few reasons. For example, some samples cannot be analyzed in typical water determining devices because the samples react with the titration reagents and/or physically interfere with components of the water determining device, such as electrodes. Therefore, these samples must be heated in an oven to drive off water. To be more specific, certain insoluble samples, such as plastics, as well as samples which react with the reagents and/or iodine, such as ascorbic acid, are typically analyzed using an oven in conjunction with either a volumetric or coulometric Karl Fischer titrator. In such a configuration, the sample is heated by the oven, and only the vaporized water is transferred to the titration cell by means of a dry inert carrier gas. In order to monitor the performance of the oven, a suitable standard must be used. Other samples are not soluble in solvents at low temperatures such as sodium chloride in methanol. Therefore, these samples must be heated to increase the solubility of the samples in the solvents. In still other scenarios, certain highly formulated petrochemical products, such as lubrication oils, may contain additives that interfere with titration, making direct analysis impossible. In such cases, an auxiliary piece of equipment, known as an oil evaporator, is used in conjunction with the titrator, such as in e.g., ASTM D6304 (Method B). Water standards are also recommended for use therein.

In these scenarios, the increase in temperature of the samples can be significant. Typically liquid water standards include hydrocarbons and/or alcohols which are not stable at high temperatures. More specifically, at temperatures above about 150° C., known liquid water standards decompose and are no longer useable. As such, accurately determining an amount of water in such samples can be difficult. Therefore, there is an opportunity for improvement and development of a liquid water standard that is stable at elevated temperatures such that the aforementioned measurements can be more easily and accurately completed.

### BRIEF SUMMARY

This disclosure provides a method of validating a water determining device. The method includes the steps of providing a liquid water standard including a room temperature ionic liquid, adding the liquid water standard to the water determining device, and determining the amount of water in the liquid water standard utilizing the water determining device thereby validating the water determining device.

### BRIEF DESCRIPTION OF THE DRAWINGS

The present disclosure will hereinafter be described in conjunction with the following drawing figures, wherein like numerals denote like elements, and

FIG. 1 is a line graph showing decomposition of a comparative (prior art) oil based water containing standard and high sample drift at elevated temperatures as set forth in the Examples;

FIG. 2 is a second line graph showing low sample drift of a liquid water standard that includes a room temperature ionic liquid as also set forth in the Examples; and

FIG. 3 is a third line graph showing low sample drift of a liquid water standard that includes a combination of a room temperature ionic liquid and a viscosity decreasing additive as also set forth in the Examples.

### DETAILED DESCRIPTION

The following detailed description is merely exemplary in nature and is not intended to limit the method or reagent. Furthermore, there is no intention to be bound by any theory presented in the preceding background or the following detailed description.

Embodiments of the present disclosure are generally directed to methods of validating a water determining device and standards for the same. For the sake of brevity, conventional techniques may not be described in detail herein. Moreover, the various tasks and process steps described herein may be incorporated into a more comprehensive procedure or process having additional steps or functionality not described in detail herein. In particular, various steps in of such validation methods are well-known and so, in the interest of brevity, many conventional steps will only be mentioned briefly herein or will be omitted entirely without providing the well-known process details. Various desirable features and characteristics of the present disclosure will become apparent from the subsequent detailed description of the disclosure and the appended claims, taken in conjunction with the accompanying drawings and the background of the disclosure.

This disclosure provides a method of validating a water determining device. The terminology “validating” may be described as standardizing the water determining device by determining a deviation from a standard (such as a liquid water standard) so as to ascertain the proper correction factors or as adjusting the water determining device precisely so that the water determining device can function properly and accurately determine an amount of water in a sample. Alternatively, the method may be described as a method of calibrating a water determining device.

### Water Determining Device:

The water determining device itself is not particularly limited any may be any known in the art. For example, the water determining device may be described as a titration device. The water determining device may be a coulometric water determining device, a volumetric water determining device, a Karl Fischer water determining device, a thermo-coulometric water determining device, a relative humidity water determining device, or a near infrared water determining device. Alternatively, the water determining device may be any used for acid-base titrations, redox titrations, gas phase titrations, complexometric titrations, zeta potential titrations, or assay titrations.

### Liquid Water Standard:

The method includes the steps of providing a liquid water standard including a room temperature ionic liquid. The step of providing is not particularly limited and may be described as supplying the liquid water standard or otherwise making the liquid water standard available for use. The liquid water standard is typically described as a reference standard or titration standard or a validation standard.

The liquid water standard may include any amount of water therein. For example, in various embodiments, the liquid water standard includes a water content of at least about 10, 50, 100, 500, 1000, 5000, or 10,000 parts by weight of water per one million parts by weight of the liquid water standard. In other embodiments, the liquid water standard includes at least about 0.1, 0.5, 1, 2, 3, 4, 5, 6, 7, 8, 9, 10, 15, or 20, weight percent of water based on a total weight of the liquid water standard. It is also contemplated that the liquid water standard may include about 25, 30, 35, 40, 45, 50, 55, 60, 65, 70, 75, 80, 85, 90, 95, or up to about 99, weight percent of water based on a total weight of the liquid water standard.

The liquid water standard may include a single room temperature ionic liquid or may include two or more room temperature ionic liquids. The room temperature ionic liquid may be any known in the art. Typically, ionic liquids are described as salt in the liquid state. The terminology “room temperature ionic liquids” typically describe that the ionic liquid is liquid at room temperature, e.g. about 20 to about 30, or about 25 to about 27, ° C., while at atmospheric pressure. While ordinary liquids predominantly include electrically neutral molecules, ionic liquids include mostly ions and short-lived ion pairs and may be described as liquid electrolytes, ionic melts, ionic fluids, fused salts, liquid salts, or ionic glasses. Some ionic liquids are electrically conducting fluids (electrolytes). Other ionic liquids can be described as moderate to poor conductors of electricity, non-ionizing (e.g. non-polar), highly viscous, and frequently exhibiting low vapor pressure. Some ionic liquids have low combustibility, thermally stability, and favorable solvating properties for a range of polar and non-polar compounds. The miscibility of ionic liquids with water or organic solvents varies with side chain lengths on cations and with choice of anion. Ionic liquids can be functionalized to act as acids, bases, or ligands.

In various embodiments, room temperature ionic liquids include bulky and asymmetric organic cations such as 1-alkyl-3-methylimidazolium, 1-alkylpyridinium, N-methyl-N-alkylpyrrolidinium and ammonium ions. Phosphonium cations are less common, but offer some advantageous properties. A wide range of anions can be utilized such as simple halides, which generally suffer high melting points, inorganic anions such as tetrafluoroborate and hexafluorophosphate, and large organic anions like bistriflimide, triflate or tosylate. Simple non-halogenated organic anions such as formate, alkylsulfate, alkylphosphate or glycolate may also be used. In various embodiments, the room temperature ionic liquid includes 1-butyl-3-methylimidazolium tetrafluoroborate, N-methyl-N-alkylpyrrolidinium cations, and/or fluorosulfonyl-trifluoromethanesulfonylimide. In other embodiments, the room temperature ionic liquid is chosen from 1,3-dimethylimidazolium methyl sulfate, 1-butyl-3-methylimidazolium methyl sulfate, 1-ethyl-3-methylimidazolium methyl sulfate, and combinations thereof.

The room temperature ionic liquid may be present in the liquid water standard in any amount and may be a balance to the water. Typically, the room temperature ionic liquid is present in an amount of about 5, 10, 15, 20, 25, 30, 35, 40, 45, 50, 55, 60, 65, 70, 75, 80, 85, 90, 95, 96, 97, 98, 99, or approximately 100, weight percent based on a total weight of the liquid water standard. If the known amount of water is present in a ppm quantity, then the room temperature ionic liquid may be described as a “balance” of the liquid water standard so as to equal a total of 100 weight percent. Accordingly, the liquid water standard may be, include, consist essentially of, or consist of, the room temperature ionic liquid (including the known amount of water). In various embodiments, such as in various “consisting essentially of” embodiments, the liquid water standard may include, or be free of, or include less than, 50, 45, 40, 35, 30, 25, 20, 15, 10, 5, 4, 3, 2, 1, 0.5, or 0.1, weight percent of a solvent, such as a protic or aprotic solvent, propylene carbonate, acetonitrile, alcohols, organic acids, hydrocarbons, ethers, halogenated hydrocarbons, carbamides, carbonates, and combinations thereof. In various non-limiting embodiments, all values and ranges of values including and between those set forth above are herein expressly contemplated for use.

In other embodiments, the liquid water standard includes a viscosity decreasing additive. The viscosity decreasing additive may be any known in the art. For example, the viscosity decreasing additive may be chosen from propylene carbonate, acetonitrile, and combinations thereof. In other embodiments, the viscosity decreasing additive is a protic or aprotic solvent or a combination thereof. In further embodiments, the viscosity decreasing additive is a protic solvent chosen from alcohols, organic acids, or combinations thereof. In other embodiments, the viscosity decreasing additive is an aprotic solvent chosen from hydrocarbons, ethers, halogenated hydrocarbons, carbamides, carbonates, and combinations thereof.

The viscosity decreasing additive and the room temperature ionic liquid may be present in any amount relative to each other. In various embodiments, the viscosity decreasing additive and the room temperature ionic liquid are present in a weight ratio of from about 1:99 to about 50:50, respectively, or about 1:99 to about 20:80, respectively. In various non-limiting embodiments, all values and ranges of values including and between those set forth above are herein expressly contemplated for use.

In other embodiments, the liquid water standard includes one or more organic and/or inorganic salts. These salts may be any known in the art and may include alkaline and/or alkaline earth metal salts, transition metal salts, etc. These salts may alternatively include organic salts such as acetates, glutamates, phosphinates, urates, diazonium salts, oxalates, tartrates, organophosphates, organosulfates, organo aminates, etc. and combinations thereof. In various embodiments, the one or more organic and/or inorganic salts are present in an amount of from about 0.1 to about 95, about 0.1 to about 5, about 5 to about 10, about 10 to about 20, or about 20 to about 95 weight percent based on a total weight of the liquid water standard. In various non-limiting embodiments, all values and ranges of values including and between those set forth above are herein expressly contemplated for use.

### Adding the Liquid Water Standard to the Water Determining Device:

Referring back, the method also includes the step of adding the liquid water standard to the water determining device. Typically, the liquid water standard is added to the water determining device as a reference or validation standard. The liquid water standard may be added or utilized in any technique or method known in the art to be used with water determining devices, such as titration devices.

### Determining the Amount of Water in the Liquid Water Standard:

The method also includes the step of determining the amount of water in the liquid water standard utilizing the water determining device thereby validating the water determining device. This step may be alternatively described as using the liquid water standard to verify an amount of water in the liquid water standard thereby validating the water determining device. This step may be completed using any method known in the art. For example, this step may be further defined as titrating. Alternatively, this step may be further defined as titrating using a coulometric water determining device, a volumetric water determining device, a thermo-coulometric water determining device, etc. This step may alternatively be described as using a relative humidity water determining device or a near infrared water determining device, using any protocol known in the art. Moreover, this step may be further defined as utilizing any one or more steps known by those of skill in the art to be typically associated with a Karl Fischer water determining device, a thermo-coulometric water determining device, a relative humidity water determining device, or a near infrared water determining device.

In various embodiments, the water determining device includes an oven. For example, for compounds that release water slowly or only at high temperatures (for example, plastics or inorganic salts), direct Karl Fischer titration methods are not suitable. Other samples have a low solubility in alcohols, which may involve toxic solvents to promote dissolution and/or involve extensive sample preparation. Introducing these samples directly into a titration cell may contaminate the cell, and thus require exchanging the titration solution and frequent cleaning of the cell. There are still other substances that undergo side reactions with the Karl Fischer reagents (for example, ascorbic acid), which lead to false results. These problems can be avoided or minimized by using an oven. First, a sample is heated in an oven and a carrier gas transfers the released water to the titration cell, where it is then determined by Karl Fischer titration. Since only the water enters the Karl Fischer cell and the sample itself does not come into contact with the Karl Fischer reagent, unwanted side reactions and matrix effects are eliminated. The sample may be introduced into the oven using a sample boat or a vial technique. For example, the sample may be weighed directly into a small sample vial, sealed with a septum cap and transferred to the oven. This method produces several advantages: strictly reproducible conditions yield results with improved precision; manual sample preparation is minimized; no contamination of the oven or titration cell by the sample is seen; and reagent consumption is diminished since the solvent is exchanged less frequently. For such samples, it is typically important to validate or calibrate the water determining device at temperatures that are going to be used to evaluate the desired sample. Accordingly, this is where the liquid water standard of this disclosure can play an important role because it is stable at elevated temperatures and thus can be used to validate the water determining device at high temperatures such that the validated or calibrated device can then be used to determine an amount of water in an experimental sample.

In various embodiments, wherein the water determining device includes an oven, the method further includes the step of heating the liquid water standard in the oven at a temperature of from about 25 to about 400, about 50 to about 350, about 100 to about 300, about 150 to about 250, or about 200 to about 250, ° C. The step of heating may occur for any time but typically for a time of from about 1 to about 800, about 1 to about 10, about 10 to about 60, about 60 to about 400, or about 400 to about 800, minutes. It is also contemplated that the sample or the liquid water standard may be heated isothermally at a temperature of from about 25 to about 400, about 50 to about 350, about 100 to about 300, about 150 to about 250, or about 200 to about 250, ° C. In various non-limiting embodiments, all values and ranges of values including and between those set forth above are herein expressly contemplated for use.

In this method, the liquid water standard may be described as a sample that is being evaluated to determine an amount of water therein. For example, the liquid water standard may be utilized as a sample that is titrated or otherwise measured to determine an amount of water therein such that the water determining device is then calibrated or adjusted so that its output matches the amount of water known to be present in the liquid water standard.

### Additional Embodiment

This disclosure also provides a method of using the liquid water standard to determine an amount of water in a further experimental sample. In such a method, the further experimental sample is not the liquid water sample but instead is an independent experimental sample. This method incorporates the steps of the aforementioned method of providing the liquid water standard, adding the liquid water standard to the water determining device, and using the liquid water standard to verify an amount of water in the liquid water standard thereby validating the water determining device. This method may also include any one or more optional steps or components described above. In addition, this method then also includes the step of utilizing the validated/calibrated water determining device to determine an amount of water in the experimental sample. In other words, this method includes validating or calibrating the water determining device and then using the validated/calibrated device to determine an amount of water in the experimental sample. The experimental sample may include any compound known in the art including, but not limited to, any samples described above that typically require use of elevated temperatures, e.g. for Karl Fischer titration. Again, the water determining device and the actual parameters of the step of determining an amount of water in the sample may be any described above and/or known in the art.

### EXAMPLES

Without intending to be bound by theory, it is believed that no liquid water standards are commercially available for use in elevated oven temperatures. Typically, liquid water standards include hydrocarbons or alcohols that are not stable at high temperatures and which decompose at temperatures above 100° C. As such, they cannot be used at elevated temperatures.

An oil based water containing standard is evaluated to determine stability at elevated temperatures and to determine drift of the water determining device. More specifically, a 3.10253 g sample of liquid mineral oil is used and includes approximately 20 ppm of water. This liquid mineral oil is commercially available from Honeywell under the tradename of Hydranal-Water Standard Oil and is introduced into an 832 KF Thermoprep Karl Fischer titration apparatus from Metrohm that is connected to a 831 KF Coulometer also from Metrohm.

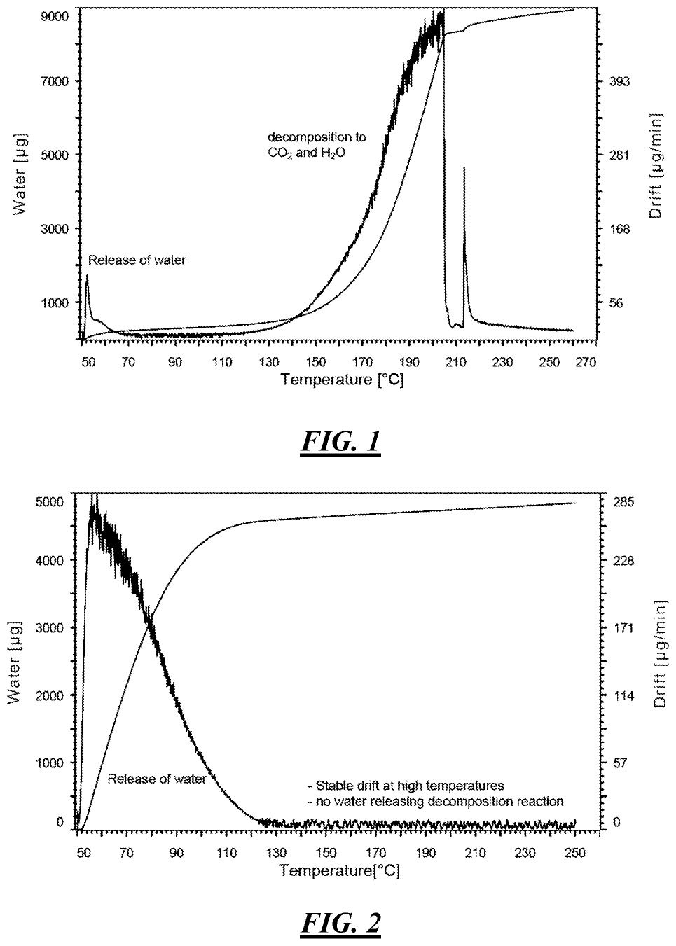

The temperature is ramped at 2° C./min from about 50° C. to about 250° C. As shown in FIG. 1, an initial peak is seen which represents a release of water in micrograms of water per minute. A second peak is indicative of an accumulated amount of water released in micrograms. Subsequently, at a temperature of about 110° C., the mineral oil begins to break down into carbon dioxide and water which leads to a dramatic increase in drift. The breakdown maximizes at about 200° C. The drift of the water determining device shows that the oil based water containing standard is not useable above about 100° C.-110° C. because data reported at such temperatures would be inaccurate.

A first liquid water standard according to this disclosure includes a room temperature ionic liquid and is also evaluated to determine stability at elevated temperatures and drift of the water determining device. More specifically, a 0.15442 g sample of 1,3-dimethylimidazolium methyl sulfate is introduced into the Karl Fischer titration apparatus described above. This room temperature ionic liquid includes approximately 3% of water. The temperature is ramped at 2° C./min from about 50° C. to about 250° C. As shown in FIG. 2, an initial peak is seen which represents a release of water at temperatures from about 50° C. to about 130° C. Subsequently, at a temperature of about 130° C., the drift becomes small and stable and no water releasing decompositions reactions are observed, in direct contrast to the decomposition of the oil based water containing standard described above and shown in FIG. 1. The low and stable drift of the water determining device observed when using this first liquid water standard is excellent, surprising, and superior to what is observed above relative to the oil based water containing standard. This means that the liquid water standard that includes the room temperature ionic liquid can be used to validate/calibrate water determining devices at high temperatures.

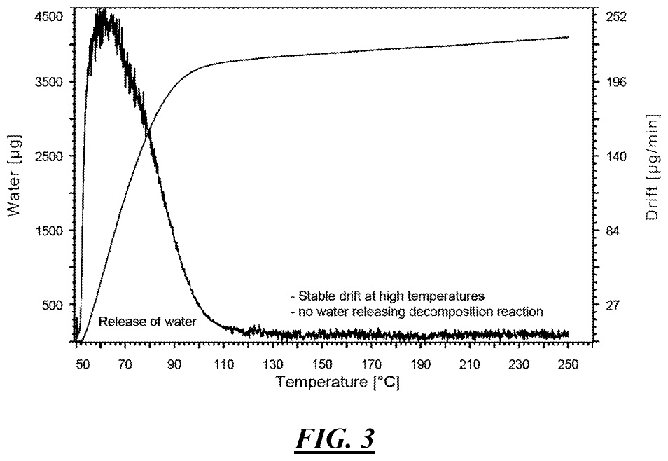

A second liquid water standard according to this disclosure includes a room temperature ionic liquid and a viscosity decreasing additive and is also evaluated to determine stability at elevated temperatures and drift of the water determining device. More specifically, a 0.13636 g sample of an 80:20 weight ratio of 1,3-dimethylimidazolium methyl sulfate and propylene carbonate is introduced into the Karl Fischer titration apparatus described above. This room temperature ionic liquid includes approximately 3% of water. The temperature is ramped at 2° C./min from about 50° C. to about 250° C. As shown in FIG. 3, an initial peak is seen which represents a release of water. Subsequently, at a temperature of about 115° C., the drift becomes small and stable and no water releasing decompositions reactions are observed, in direct contrast to the decomposition of the oil based water containing standard described above and shown in FIG. 1. The low and stable drift of the water determining device observed when using this second liquid water standard is excellent, surprising, and superior to what is observed above relative to the oil based water containing standard described above and shown in FIG. 1. This means that the liquid water standard that includes the room temperature ionic liquid and the viscosity decreasing additive can be used to validate/calibrate water determining devices at high temperatures.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration in any way. Rather, the foregoing detailed description will provide those skilled in the art with a convenient road map for implementing an exemplary embodiment. It being understood that various changes may be made in the function and arrangement of elements described in an exemplary embodiment without departing from the scope as set forth in the appended claims.

What is claimed is:

1. A method of validating a water determining device, said method comprising the steps of:
A. providing a liquid water standard comprising a room temperature ionic liquid;
B. adding the liquid water standard to the water determining device; and
C. determining the amount of water in the liquid water standard utilizing the water determining device thereby validating the water determining device.

2. The method of claim 1 wherein the liquid water standard has a water content of at least 10 parts by weight of water per one million parts by weight of the liquid water standard.

3. The method of claim 1 wherein the liquid water standard has a water content of at least 1 weight percent of water based on a total weight of the liquid water standard.

4. The method of claim 1 wherein the water determining device comprises an oven and the method further comprises the step of heating the liquid water standard in the oven at a temperature of from about 25° C. to about 400° C.

5. The method of claim 1 wherein the water determining device comprises an oven and the method further comprises the step of heating the liquid water standard in the oven at a temperature of from greater than about 100° C. to a temperature of about 400° C.

6. The method of claim 1 wherein the room temperature ionic liquid is chosen from 1,3-dimethylimidazolium methyl sulfate, 1-butyl-3-methylimidazolium methyl sulfate, 1-ethyl-3-methylimidazolium methyl sulfate, and combinations thereof.

7. The method of claim 1 wherein the liquid water standard is free of a solvent.

8. The method of claim 1 wherein the liquid water standard is free of hydrocarbons and alcohols.

9. The method of claim 1 wherein the liquid water standard further comprises a viscosity decreasing additive.

10. The method of claim 9 wherein the viscosity decreasing additive is chosen from propylene carbonate, acetonitrile, and combinations thereof.

11. The method of claim 9 wherein the viscosity decreasing additive is a protic or aprotic solvent or a combination thereof.

12. The method of claim 11 wherein the viscosity decreasing additive is a protic solvent chosen from alcohols, organic acids, or combinations thereof.

13. The method of claim 11 wherein the viscosity decreasing additive is an aprotic solvent chosen from hydrocarbons, ethers, halogenated hydrocarbons, carbamides, carbonates, and combinations thereof.

14. The method of claim 9 wherein the viscosity decreasing additive and the room temperature ionic liquid are present in a weight ratio of from about 1:99 to about 50:50, respectively.

15. The method of claim 9 wherein the viscosity decreasing additive and the room temperature ionic liquid are present in a weight ratio of from about 1:99 to about 20:80, respectively.

16. The method of claim 9 wherein the liquid water standard comprises two or more room temperature ionic liquids.

17. The method of claim 16 wherein the liquid water standard comprises one or more organic and/or inorganic salts.

18. The method of claim 1 wherein the liquid water standard comprises two or more room temperature ionic liquids.

19. The method of claim 18 wherein the liquid water standard comprises one or more organic and/or inorganic salts.

20. The method of claim 1 wherein the water determining device is chosen from a coulometric water determining device, a volumetric water determining device, a thermo-coulometric water determining device, a relative humidity water determining device, or a near infrared water determining device.
